(19) United States
(12) Patent Application Publication
(10) Pub. No.: US 2004/0087563 A1
(43) Pub. Date: May 6, 2004

(54) Hormone replacement therapy with cardiovascular protection using antialdosteronic progestins

(76) Inventor: Siegfried Mayerhofer, Vienna (AT)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/287,780

(22) Filed: Nov. 5, 2002

(51) Int. Cl. A61K 31/57

(52) U.S. Cl. 514/177

(57) ABSTRACT

The present invention relates to methods of treating aldosterone-mediated diseases such as cardiovascular diseases in peri-menopausal or post-menopausal women by administering an agent with antimineralocorticoid and progestational activity, in particular drospirenone.

### FIELD OF INVENTION

The present invention relates to a pharmaceutical composition comprising drospirenone as the sole therapeutically active agent and to methods of treating symptoms and diseases related to deficient levels of progesterone, in particularly to methods of simulataneous treating symptoms and diseases related to deficient levels of progesterone and aldosterone-mediated diseases such as cardiovascular diseases.

### BACKGROUND

A number of mechanisms are now recognised in the pathogenesis of cardiovasculary diseases. For long it has been the understanding that the predominant mechanism of preventing hypertension was through maintaining electrolyte homeostasis via the renin-angiotensin-aldosterone system (RAAS) in that the plasma levels of the mineralocorticoid hormone, aldosterone, was reduced by inhibiting the angiotensin converting enzyme. Aldosterone has been known to exert its activities through activation of mineralocorticoid receptors present in the epithelia of the kidney, colon and sweat glands whereby aldosterone promote the retention of sodium and the excretion of potassium. One of the most widely used agents for reducing hypertension is the so-called ACE inhibitors that act through inhibition of the angiotensin-converting enzyme.

Hypertension has for many years been thought to be the pre-dominant factor in the development of cardiovascular diseases, but it has now been questioned to what extent ACE inhibitors prevents the development of cardiovascular diseases.

However, recently it was shown that mineralocorticoid receptors are also present in the cardiovascular system, the heart and blood vessels, as well as in the brain (Delayani J A: Mineralocorticoid receptor antagonists; the evolution of utility and pharmacology. Kidney INt, 2000 April 57(4) 1408-1411). Moreover, animal and human studies points to the fact that aldosterone plays a role in the pathogenesis of cardiovascular and renal diseases independent of the well-known mechanism involving activation of the angiotensin to angiotensin II (Stier et al, aldosterone as a mediator in cardiovascular injury. Cardiol Rev 2002, March-April 10(2), 97-102). Thus, it is anticipated that aldosterone via its activation of the mineralocorticoid receptors in the hearth, blood vessels or brain is capable of mediating several pathophysiological actions like for example stroke, cardiac fibrosis, ventricular hypertrophy, myocardial necrosis, heart failure (congestive heart failure), sudden cardiac death and/or myocardial infarction.

In women the risk of developing cardiovascular diseases increases after entering the menopause, where ovulation stops (Aflalo-Calderon B. HRT, women, and hearth disease: what we need to know about prevention. Medscape Cardiology 6(2), 2002). The rationale for establishing hormone replacement therapy in post-menopausal women with estrogens or with the combination of an estrogen and a progestin has for many years been related to the alleviation or preventing of those symptoms or diseases that result from deficient endogenous levels of estrogen, such as hot flushes. However, an important goal with hormone replacement therapy was to provide reduced risk of developing diseases that are thought or known to occur more frequently in women experiencing low endogenous levels of estrogen. Of importance Is reduced risk of osteoporosis and/or cardiovascular diseases. While the beneficial effect of hormone replacement therapy with estrogens or combinations of estrogens and progestins on osteoporosis is well established, the risk of developing cardiovascular diseases by the current forms of therapy has not yet been verified (Nelson et al. Postmenopausal hormone replacement therapy. J Am Medical Ass, Aug. 21, 2002, vol 288, no 7, pp 872-891). It is currently considered problematic that the hormone replacement therapy used in the clinic today does not reduce the risk of cardiovascular diseases.

It has long been considered that deficient levels of estrogen accounted for the increased risk of developing cardiovascular diseases in post-menopausal women. However, deficient endogenous levels of progesterone may in theory also play a role in the increased risk of developing cardiovascular diseases in post-menopausal women.

The normally occurring levels of endogenous progesterone start to decline in the years before menopause. The average age of the onset of menopause is 51 years, and it is most commonly present in the age from about 47 to 53. The period immediately before and after the onset of menopause is termed peri-menopause, and averages about 4 years. Peri-menopause is characterised by irregular and abnormal bleeding, which can be controlled by treatment with progesterone.

A number of agents with aldosterone receptor blocker activity have been developed for treating aldosterone-mediated diseases such as cardiovascular diseases. Of importance are substances possessing selective binding to the mineralocorticoid receptor in that they do not affect other steroidal receptors such as the estrogen and progesterone receptors.

Eplerenone is an example of such a drug(Martin and Krum; Eplerenone. Current opinion in investigational drugs, 2001, 2(4), 521-525). Eplerenone has beneficial cardioprotection (Hamedii and Chadow: The promise of selective aldosterone receptor antagonist for the treatment of hypertension and congestive hearth failure. Curr Hypertens Rep, 2000, August 2(4), 378-383, Delayani et al: Eplerenone; a selective aldosterone receptor antagonist (SARA), cardiovascular Drug Rev 2001 FAll, 19(3)185-200).

However, for the purpose of reducing the risk of cardiovascular diseases in women such as in women in peri-menopause and post-menopause, it is advantageously to use a therapeutically active agent with combined antagonism of the mineralocorticoid receptor and progestational effect, optionally in combination with an estrogen.

One such agent with combined anti-aldosterone and progestational effect is drospirenone (DRSP), a 17α-spirolactone and an analog to spirolactone. Drospirenone has unlike other known progestins biochemical and pharmacological profiles similar to endogenous progesterone, especially with regard to anti-mineralocorticoid and anti-androgenic activities (Krattenmacher R; drospirenone: pharmacology and pharmacokinetics of a unique progestogen. Contraception 2000, July, 62(1), 29-38; Norman P, Drospirenone. Drugs of the future 2000, 25(12, 1247-1256). Therefore, DRSP can mediate the natural anti-aldosterone and vasoactive properties of endogenous progesterone in women. Drospirenone binds to the mineralocorticoid receptor in competition with aldosterone so as to provide a strong antimineralocorticoid activity. Drospirenone is about 8 times as potent as spironolactone (Pollow et al; dihydrospirorenone, a novel synthetic progestagen, characterisation of binding to different receptor proteins. Contraception, 1992, 46, 561-574).

Drospirenone is known from the patent DE 19633685 and the patent application WO 98067838 that both relates to a process for producing drospirenone, 6β,7β;15β;16β-dimethylene-3-oxo-17α-preg-4-ene-21, 17-carbolactone.

Plasma aldosterone levels may be affected by administering drospirenone: In a study implying administration of drospirenone alone or drospirenone in combination with ethinylestradiol demonstrated that the plasma aldosterone levels increased in both groups of therapy. (Oelkers et al: Effect of an oral contraceptive containing drospirenone in the renin-angiotensin-aldosterone system in healthy female volunteers, Gynecol endocrinol 2000, 14, 204-213).

Furthermore, it has been shown that the combination of drospirenone and 17β-estradiol further reduces hypertension in postmenopausal women that suffer from hypertension and is in therapy with an ACE inhibitor, Enalapril (Preston et al, Additive effect of drospirenone/17β-estradiol in hypertensive postmenopausal women receiving Enalapril (Am J Hypertension, 2002, 15 (816-822).

Drospirenone is further known from its use in contraception (WO 01/15701.) and for HRT (WO/52857)

Spironolactone (17-hydroxy-7-alpha-mercapto-3-oxo-17-alpha-pregn-4-ene-21-carboxylic acid gamma-lactone acetate) is used as an hypertensive and diuretic drug and belongs to the group of spirolactones, wherein drospirenone also belongs. Spironolactone has been shown to decrease the morbidity and mortality among patients with severe heart failure who were already receiving ACE inhibitor therapy. Thus, an additive effect was observed (Pitt et al; the effect of spironolactone on morbidity and mortality in patients with severe heart failure. New Engl. J Med, vol 341, no 10, pp 709717, 1999). Spironolactone is an effective aldosterone antagonist, but is thought to have considerable side effects in recommended daily dosages, e.g. increasing the risk of cancer. Spironolactone may also give major disruptions in the balance of minerals within the body.

Spironolactone has been used for the following purposes in relation to the treatment of aldosterone mediated pathogenic effects:

WO 01/95892 relates to the treatment of aldosterone mediated pathogenic effects such as cardiovascular diseases in a subject that has salt sensitivity or an elevated dietary sodium intake or both by administering one or more aldosterone antagonists such as spironolactone.

WO 02/09759 relates to the treatment of inflammation-related cardiovascular disorders such as aetherosclerosis in a subject by administering an aldosterone antagonist and a cyclooxygenase-2 inhibitor. The aldosterone antagonist being a spirolactone-type compound (spironolactone) and an epoxy-steroidal aldosterone antagonist.

WO 01/34132 relates to the treatment, inhibiting or preventing pathogenic change resulting from vascular injury in a human subject by administering an aldosterone antagonist.

WO 95/15166 relates to the treatment of an aldosterone antagonist such as spironolactone and epoxymexrenone for inhibiting myocardial fibrosis in that the dosage used may not disrupt a patients normal electrolyte and water-retention balance.

### SUMMARY OF THE INVENTION

It has been found that drospirenone, an agent with combined progestational and antimineralocorticoid activity antagonises the action of aldosterone mediated through mineralocorticoid receptors present in the hearth, blood vessels and brain. This observation has advantageous implications to the current hormone replacement therapy of peri- and post-menopause since the woman will benefit from the reduced risk of developing aldosterone-mediated disease, in particular cardiovascular diseases. Thus, agents with combined actions on progestational and mineralocorticoid receptors, in particular those agents having similar potency as drospirenone in the antagonism of aldosterone via the mineralocorticoid receptors and/or having similar potency as drospirenone in progestational actions, are anticipated by the present invention.

Accordingly, a first aspect of the invention relates to a composition for treating aldosterone-mediated diseases comprising as the sole therapeutically active agent drospirenone and/or pharmaceutically acceptable salts thereof.

Further aspects of the invention relate to the use of an agent exhibiting progestational and antimineralocorticoid activity for the preparation of a medicament for treating the conditions as mentioned below in items 1-3. Moreover, further aspects relates to a therapeutic method comprising administering an agent exhibiting progestational and antimineralocorticoid activity to a woman for treating the conditions as mentioned below in items 1-3.

1) treating symptoms, diseases or disorders associated with deficient endogenous levels of progesterone in a woman;

2) treating and/or preventing aldosterone-mediated diseases in a woman that is in need of progesterone replacement therapy; or

3) simultaneous treating or preventing symptoms associated with low endogenous levels of progesterone and aldosterone-mediated diseases in a woman.

According to the invention, it is contemplated that the beneficial effects of an agent exhibiting progestational and antimineralocorticoid activity for treating aldosterone-mediated diseases in peri and post-menopausal women may also implicate the concurrent use of an estrogen. Therefore, still further aspects of the invention relate to uses for preparation of a medicament and to therapeutic methods comprising;

4) an estrogen and an agent exhibiting progestational and antimineralocorticoid activity for simultaneous treating and/or preventing diseases, disorders and symptoms associated with deficient endogenous levels of estrogen and aldosterone-mediated diseases in a woman; or

5) an agent exhibiting progestational and antimineralocorticoid activity for treating and/or preventing aldosterone mediated diseases in a woman in need of estrogen replacement therapy.

It is further considered that the beneficial effects on aldosterone-mediated diseases in a woman in peri-menopause and post-menopause may be achieved by the combination of one agent exhibiting antimineralocorticoid effect and substantially no or low progestational activity and a second agent exhibiting substantially no antimineralocorticoid effect, but progestational effect.

Therefore, still further aspects of the invention relate to uses for preparation of a medicament and to therapeutic methods comprising;

6) a combination of a selective aldosterone antagonist and a progestin with substantially no antimineralocorticoid effect for treating and/or preventing aldosterone-mediated diseases in a woman that is in need of progesterone replacement therapy;

7) an estrogen, a selective aldosterone antagonist and a progestin with substantially no antimineralocorticoid effect for simultaneous treating and/or preventing diseases, disorders and symptoms associated with deficient endogenous levels of estrogen and aldosterone mediated diseases in a woman; or

8) a combination of a selective aldosterone antagonist and a progestin with substantially no antimineralocorticoid effect for the preparation of a medicament for treating and/or preventing aldosterone mediated diseases in a woman in need of estrogen replacement therapy.

In preferred embodiments of the invention, the agent exhibiting progestational and antimineralocorticoid activity is drospirenone.

Of importance to the invention is the dosage of drospirenone wherein it inhibits aldosterone-mediated diseases. Thus, an interesting aspect of the invention relates to uses for preparation of a medicament and to therapeutic methods comprising drospirenone in a daily deliverable dose of 0.1 to 5 mg for the preparation of a medicament for treating and/or preventing aldosterone-mediated diseases in a subject in need thereof.

### DETAILED DESCRIPTION

As used herein the following phrases and terms is defined:

The phrase “an agent exhibiting progestational and antimineralocorticoid activity” is intended to denote an agent that has a relative binding affinity to the antimineralocorticoid receptor and the progestational receptor in the order of drospirenone. A compound is characterised as “an agent exhibiting progestational and antimineralocorticoid activity” when the relative binding affinity to the progestational receptor and the relative binding affinity to the mineralocorticoid receptor as determined in relation to the binding affinity of drospirenone with respect to these two receptors is in the range of 50% to 150%, preferably in the range of 75% to 125%, more preferably in the range of 85% to 115%.

The phrase “deficient endogenous levels of progesterone” is intended to mean that the plasma concentration of progesterone has, for at least one month as determined on a daily basis, been lower than the concentration range expected during luteal phase of the menstrual cycle. The expected progesterone plasma concentration during luteal phase is in the range between 30 and 110 nmol/l. In the event of a pregnant woman, the phrase “deficient endogenous levels of progesterone” is intended to mean progesterone plasma concentrations lower than 200 nmol to 300 nmol. The plasma concentrations of progesterone in a pregnant woman rise steadily until they peak at the end of the third trimester in the range of 320-700 nmol/l.

The term “menopause” characterises the time in a woman's life when the ovaries stop producing estrogen. Menopause is usually recognised by the cessation of menstrual periods. Other symptoms of menopause include flashes, mood changes, difficulty sleeping, and vaginal dryness. In the present context, the term menopause is understood as the last natural (ovary-induced) menstruation. It is a single event and a result of an age-dependent dysfunction of the ovarian follicles. Menopause results from the ovaries decreasing their production of the sex hormones estrogen and progesterone. When the number of follicles falls below a certain threshold (a bleeding threshold), the ovaries can no longer produce mature follicles and sex hormones. The ability to reproduce capability ends with menopause. If a woman is not menstruating because she has had a hysterectomy or endometrial ablation, other symptoms of menopause often alert her that menopause is starting. The average age of the onset of menopause is 51 years, and it most commonly occurs from age 47 to 53.

The term “per-menopause” characterises the period immediately before and after the onset of menopause, and averages 4 years. Furthermore, the peri-menopause is characterised by abnormal and irregular bleeding. The peri-menopausal phase begins with the onset of climacteric symptoms when the cycle becomes irregular and ends one year after menopause. The end of peri-menopausal phase can be identified after a protracted period of time without bleeding.

The term “post-menopause” is the phase that begins at menopause and continues until death.

The term “irregular bleeding” characterises any uterine bleeding, outside the regular monthly menstrual periods of non-pregnant women. Uterine bleeding are irregular if menstrual cycles or menstrual periods are too short, too long, too frequent, too infrequent, or occur at irregular intervals which falls outside the regular 26-30 days menstrual cycle. The menstrual period is classified as too long when being delayed with 15 to 50 days or more to the expected onset of said bleeding.

The term “abnormal bleeding” characterises heavy bleeding typically soaking through enough sanitary protection products to require changing more than every one or two hours, having a period that lasts over seven days. Abnormal bleeding does not include bleeding in women who have already reached menopause, abnormal uterine bleeding due to side effects of hormone replacement therapy, abnormal bleeding as a symptom of uterine cancer, as a result of a consequence of abnormal blood clotting normally, an inherited bleeding disorder or because of a medical illness that affects levels of blood platelets.

The term “lack of ovulation” relates to the condition in a woman, where the woman has stopped ovulating. The condition is characterised by a relative blood level of luteinizing hormone greater than 30 IU/L in a peri-menopausal/menopausal woman, in comparison with a menstruating woman with a level of 5-22 IU/L or 30-250 IU/L in respectively the follicular or luteal phase or midcycle phase.

The phrase “a woman that is in need of progesterone replacement therapy” qualifies a woman that is deficient in endogenous levels of progesterone, a woman that is pregnant and in risk of miscarriage, a woman that experience irregular bleeding, abnormal bleeding.

The phrase “epoxy-steroidal aldosterone receptor antagonist” is intended to denote one or more agents characterised by a steroid-type nucleolus and an epoxy moiety attached to the nucleolus and which agent or compound binds to the aldosterone receptor as a competitive inhibitor of the action of aldosterone itself at the receptor site, so as to modulate the receptor-mediated activity of aldosterone.

The phrase “non-epoxy-steroidal aldosterone receptor antagonist” is intended to denote one or more agents characterised by a steroid-type nucleolus and with no epoxy moiety attached to the nucleolus and which agent binds to the aldosterone receptor as a competitive inhibitor of the action of aldosterone itself at the receptor site, so as to modulate the receptor-mediated activity of aldosterone.

The term “steroidal” denotes a nucleus provided by a cyclopenteno-phenathrene moiety, having the conventional four ring members.

The term “epoxy-steroidal” is intended to embrace a steroidal nucleus having one or a plurality of epoxy-type moieties attached thereto.

The term “therapeutically effective amount” is intended to qualify the amount of an agent for the use in therapy, which will achieve the goal of a wanted response in reducing, preventing or treating symptoms, conditions or disorders related to a disease, preferably while avoiding adverse side effects.

The term “preventing” includes either prevention of the onset of a clinical evident disorder altogether or preventing the onset of a preclinically evident stage of disorder in individuals.

The term “aldosterone antagonist” is denoted to include any substance that reduces the activity of aldosterone as a result of inhibiting its binding to the antimineralocorticoid receptor. It does not include substances, which reduce the amount of aldosterone synthesized or secreted by the adrenal cortex. Mespirenone is an example of an inhibitor of aldosterone synthesis.

The term “aldosterone-mediated diseases” encompasses diseases mediated through a compound binding to the aldosterone receptor site, and hereby modulating the receptor-mediated activity of aldosterone.

As used herein “spironolactone” refers to a molecule comprising a lactone structure coupled via a spiro configuration to a steroid structure or steroid derivative.

The phrase “selective aldosterone antagonist” relates to a compound that has a relative binding affinity to the antimineralocorticoid receptor and the progestational receptor in the order of eplerenone. A compound is characterised as a “selective aldosterone antagonist” when the relative binding affinity to the progestational receptor and the relative binding affinity to the mineralocorticoid receptor as determined in relation to the binding affinity of eplerenone to these two receptors is in the range of 50% to 150%, preferably in the range of 75% to 125%, more preferably in the range of 85% to 115%.

The phrase “progestin with substantially no antimineralocorticoid effect” qualifies to a compound that has a relative binding affinity to the mineralocorticoid receptor in relation to drospirenone of less than 1%, preferably of less than 0.2% such as of less than 0.01%.

The phrase “progestin with low antimineralocorticoid effect” qualifies to a compound that has a relative binding affinity to the mineralocorticoid receptor in relation to drospirenone of less than less than 10%, preferably less than 5% such as less than 2%.

The term “relative binding affinity to the progestational receptor” is characterised by the binding affinity of a test agent relatively to that of drospirenone as determined in cytosol fractions containing expression vector for a animal or human progestational receptor, respectively. The assay can be carried out incubating the cytosols with an appropriate radiolabeled reference substances, for which the relative binding affinity value is set at 100% and the steroid competitor, as described in K. Pollow et al. Contraception, 46:561-574, 1992. In the event where the test agent is tested for selective aldosterone receptor antagonism, the relative binding affinity is determined relatively to eplerenone

The term “relative binding affinity to the mineralocorticoid receptor” is characterised by the binding affinity of a test agent relatively to that of drospirenone as determined on the basis of similar doses of the two agents in cytosol fractions containing expression vector for a animal or human mineralocorticoid receptor, respectively. The assay can be carried out incubating the cytosols with an appropriate radiolabeled reference substances, for which the relative binding affinity value is set at 100% and the steroid competitor, as described in K. Pollow et al. Contraception, 46:561-574, 1992. In the event where the test agent is tested for selective aldosterone receptor antagonism, the relative binding affinity is determined relatively to eplerenone.

A compound is characterised as having low anti-mineralocorticoid effect or low progestational effect when the binding affinity to the relevant receptors of the test agent relatively to drospirenone is less than 10%, preferably less than 5% such as less than 2%.

A compound is characterised as having substantially no anti-mineralocorticoid effect or substantially no progestational effect when the binding affinity to the relevant receptors of the compound relatively to drospirenone is less than 1%, preferably less than 0.2% such as less than 0.01%.

A compound is characterised as having a selective aldosterone antagonism when the binding affinity of the compound relatively to eplerenone with regard to the progestational receptor and the mineralocorticoid receptor is in the range of 50% to 150%, preferably in the range of 75% to 125%, more preferably In the range of 85% to 115%.

A compound is characterised as having a progestational and anti-mineralocorticoid effect similar to drospirenone when the binding affinity of the compound relatively to drospirenone with regard to the progestational receptor and the mineralocorticoid receptor is in the range of 50% to 150%, preferably in the range of 75% to 125%, more preferably in the range of 85% to 115%.

The term “derivatives” is intended to encompass esters, ethers, products or salts.

As stated, the invention lies in part in the beneficial dual action of drospirenone and other agents with a pharmacological and biochemical profile similar to drospirenone in the treatment of symptoms, diseases and disorders associated with deficient endogenous levels of progesterone in a woman, preferably in a dosage wherein the woman has a reduced risk of developing aldosterone-mediated diseases such as cardiovascular diseases. Importantly, this action is mediated with drospirenone as the sole therapeutically active agent. Treatment of women having low endogenous levels of progesterone with an agent possessing combined progestational and antimineralocorticoid effect may be of benefit to those women who are susceptible to aldosterone-mediated diseases such as cardiovascular diseases.

Accordingly, a first aspect of the invention relates to a composition comprising as the sole therapeutically active agent drospirenone or derivatives thereof, and optionally pharmaceutical excipient(s) or carrier(s). According to the present invention, drospirenone is intended to include any geometric isomer of drospirenone, metabolites of drospirenone and/or derivatives of drospirenone as long as they exhibit the same pharmacological and biochemical actions as drospirenone itself. That is to say that the isomers, metabolites or derivatives of drospirenone may have a progestational and anti-mineralocorticoid effect similar to drospirenone when the binding affinity of the test agent relatively to drospirenone with regard to the progestational receptor and the mineralocorticoid receptor is in the range of 50% to 150%, preferably in the range of 75% to 125%. Drospirenone may be in the form of an ester or prodrug such as an oxyiminopregnane carbolactone as disclosed in WO 98/24801.

A second aspect of the invention relates to the treatment of deficient endogenous levels of progesterone in a woman such as to the use of an agent exhibiting progestational and antimineralocorticoid activity for the preparation of a medicament for treating symptoms, diseases or disorders associated with deficient endogenous levels of progesterone in a woman. Preferably, such an agent is the sole therapeutically active agent and the agent is preferably drospirenone.

That is to say that the second aspect of the invention relates to a therapeutic method for treating, alleviating or preventing symptoms, diseases or disorders associated with deficient endogenous levels of progesterone comprising administering to a woman an agent exhibiting progestational and antimineralocorticoid activity. In current preferable embodiments, the agent exhibiting progestational and antimineralocorticoid activity is the sole therapeutically active agent.

Deficient endogenous levels of progesterone may be found in a woman for a number of reasons. Mainly, deficient endogenous levels of progesterone can be depicted in a woman experiencing irregular bleeding, abnormal bleeding, lack of ovulation, peri-menopause or post-menopause. Also a pregnant woman may suffer from deficient endogenous levels of progesterone in that a pregnant woman normally will have high levels of progesterone in order to avoid risk of miscarriage. Furthermore, deficient endogenous levels of progesterone are related to endometriose and other gynecological disorders.

Therefore, the invention relates to the treatment and/or prevention of irregular bleeding, abnormal bleeding, lack of ovulation, peri-menopause, post-menopause, risk of miscarriage, endometriose and/or other gynecological disorders.

In preferred embodiments of the invention, the uses and therapeutic methods are preferably directed to a peri-menopausal woman.

Importantly, the uses and methods of therapy of the invention are also directed to a woman susceptible to aldosterone-mediated diseases such as cardiovascular diseases. Such women may be healthy but nonetheless being in the risk of developing cardiovascular diseases, in particular upon experiencing deficient endogenous levels of progesterone, such as a woman in peri-menopause or in post-menopause, or such as a pregnant woman in risk of miscarriage or hypertension.

Accordingly, other aspects of the invention relates to uses for preparation of a medicament and to therapeutic methods comprising an agent exhibiting progestational and antimineralocorticoid activity for treating and/or preventing aldosterone-mediated diseases in a woman that is in need of progesterone replacement therapy.

The dosage of the agent exhibiting progestational and antimineralocorticoid activity that is effective in treating and/or preventing aldosterone-mediated diseases in a woman, such as achieving reduced risk of cardiovascular diseases is important to the invention. For example the agent exhibiting progestational and antimineralocorticoid activity may be used in a dosage that are below of the dosage resulting in treating symptoms associated with peri-menopause, post-menopause, prevention of miscarriage in a pregnant woman, prevention of hypertension of a pregnant woman. In that event, the dosage of the agent exhibiting progestational and antimineralocorticoid activity may be effective in reducing the risk of developing aldosterone-mediated diseases arising from aldosterone antagonism at the mineralocorticoid receptor, but not effective in treating symptoms and diseases associated with deficiencies in the endogenous levels of progesterone in a woman. Thus in those embodiments, the dosage does not result in regular bleeding (cycle control) and/or normal bleeding in a woman or prevention of miscarriage in a pregnant woman. In such embodiments, the uses and therapeutic methods of the invention include that the woman is in concurrent therapy with one or more alternative progestin(s) in dosages that will provide regular bleeding (cycle control), normal bleeding, treat endometriose and/or treat other gynecological disorders in a woman, or prevention of miscarriage in a pregnant woman. Without limitations, such alternative progestins may include those having low or substantially no aldosterone receptor blocker effect/antimineralocorticoid effect in comparison to drospirenone. Such progestins are characterised by having low antimineralocorticoid effect.

However, in some interesting aspects of the invention it is advantageous to use a dosage of the agent exhibiting progestational and antimineralocorticoid activity that provides beneficial effects to the cardiovascular system while simultaneous treating deficiencies in the endogenous levels of progesterone in a woman.

Hence, a still further aspect of the invention relates to uses for preparation of a medicament and to therapeutic methods comprising an agent exhibiting progestational and antimineralocorticoid activity for simultaneous treating and/or preventing symptoms associated with low endogenous levels of progesterone and aldosterone-mediated diseases in a woman.

As mentioned, low endogenous levels of progesterone are an indication of peri-menopause in a woman. However, post-menopausal women does also have low endogenous levels of progesterone or even lack of progesterone for which reason post-menopausal women may have beneficial effect upon administering an agent exhibiting progestational and antimineralocorticoid activity. However, post-menopausal women are often also in need of therapy with estrogens. Therefore, in further embodiments of the invention, the woman that is in need of progesterone replacement therapy, e.g. a woman with deficient levels of endogenous progesterone may be in concurrent therapy with estrogens. This relates for example to a woman that has entered the post-menopause period, where the production of estrogens in the ovaries has stopped.

Accordingly, a still further aspect of the invention relates to uses for preparation of a medicament and to therapeutic methods comprising an agent exhibiting progestational and antimineralocorticoid activity for treating and/or preventing aldosterone mediated diseases in a woman in need of estrogen replacement therapy

In some related aspects thereto, the medicament may comprise an estrogen and the therapeutic method may comprise concurrent administering of an estrogen. Thus, a further aspect of the invention relates to uses for preparation of a medicament and to therapeutic methods comprising an agent exhibiting progestational and antimineralocorticoid activity and one or more estrogen(s) for simultaneous treating symptoms, diseases or disorders associated with deficient endogenous levels of estrogen and aldosterone-mediated diseases in a woman.

In preferred embodiments, the estrogen is selected from the group consisting of estradiol, estradiol sulfamates, estradiol valerate, estradiol benzoate, ethinyl estradiol, estrone, estriol, estriol succinate and conjugated estrogens, including conjugated equine estrogens such as estrone sulfate, 17β-estradiol sulfate, 17α-estradiol sulfate, equilin sulfate, 17β-dihydroequilin sulfate, 17α-dihydroequilin sulfate, equilenin sulfate, 17α-dihydroequilenin sulfate and 17α-dihydroequilenin sulfate or mixtures thereof. Particularly interesting estrogens are selected from the group consisting of estradiol, estradiol sulfamates, estradiol valerate, estradiol benzoate, estrone, and estrone sulfate or mixtures thereof, notably estradiol, estradiol valerate, estradiol benzoate and estradiol sulfamates. Most preferred are estradiol or estradiol sulfamates, particularly estradiol.

The deficient levels of estrogen may be caused by natural menopause, peri-menopause, post-menopause, hypogonadism, castration or primary ovarian failure.

The diseases, disorders and symptoms may be any of or a mixture of the following conditions: hot flushes, sweating attacks, palpitations, sleep disorders, mood changes, nervousness, anxiety, poor memory, loss of confidence, loss of libido, poor concentration, diminished energy, diminished drive, irritability, urogenital atrophy, atrophy of the breasts, cardiovascular disease, changes in hair distribution, thickness of hair, changes in skin condition and/or osteoporosis.

According to the present invention, the agent exhibiting progestational and antimineralocorticoid activity may be a non-epoxy spironolactone-type steroidal compound such as spironolactone or drospirenone. The non-epoxy spironolactone-type steroidal compound may further be characterised by having a relative binding affinity to the mineralocorticoid receptor and the progestational receptor of substantially the same order as drospirenone. In current preferred embodiments the agent exhibiting progestational and antimineralocorticoid activity is drospirenone. A number of side-effects among them serious effects such as cancer has been reported for Spironolactone when used in therapeutically active doses. Moreover, the progestational activity of spironolactone is far lower than that of drospirenone and the progestational effective dosage of spironolactone may disrupt a patients normal electrolyte and water-retention balance. Therefore, it is anticipated that drospirenone is advantageous in that it has dual actions (progestational and antagonism of aldosterone-mediated diseases) within the same dosage range. Furthermore, in some dosages drospirenone may antagonise aldosterone-mediated diseases without affecting the progestational activity.

As stated, an evident feature of the present invention include an therapeutically active agent for treating aldosterone-mediated diseases in for example peri- and post-menopausal women in that such an agent should be capable of providing both progestational and antimineralocorticoid activity. However, in some aspects of the invention such a combined activity may result from the combination of a selective aldosterone antagonist and a progestin with low or substantially no antimineralocorticoid effect.

Hence, further aspects of the invention relates to uses for preparation of a medicament and to therapeutic methods comprising a combination of a selective aldosterone antagonist and a progestin with substantially no or low antimineralocorticoid effect for treating and/or preventing aldosterone-mediated diseases in a woman that is in need of progesterone replacement therapy. In the event where the woman also is in need of estrogens, a still further aspect relates to uses for preparation of a medicament and to therapeutic methods comprising a combination of a selective aldosterone antagonist and a progestin with substantially no or low antimineralocorticoid effect and an estrogen for simultaneous treating and/or preventing diseases, disorders and symptoms associated with deficient endogenous levels of estrogen and aldosterone mediated diseases in a woman. Still another aspect relates to uses for preparation of a medicament and to therapeutic methods comprising a combination of a selective aldosterone antagonist and a progestin with substantially no or low antimineralocorticoid effect for treating and/or preventing aldosterone mediated diseases in a woman in need of estrogen replacement therapy.

The selective aldosterone antagonist may be an epoxy spironolactone-type steroidal compound such as eplerenone (epoxymexrenone) or canrenoate.

A great number of progestins with substantially no or low antimineralocorticoid effect are known. Examples are, but not limited to norethisterone; norethisterone acetate; levonorgestrel; gestodene; norgestilmate; dienogest; medroxyprogesterone acetate; megestrol acetate; chlormadinone acetate; or cyproterone acetate. The progestins may be in the form of its derivates such as esters, ethers or salts, preferably esters. Progesterone may have a lower antimineralocorticoid effect than drospirenone and eplerenone, in particular at those doses that are clinically relevant for exhibiting progestational effect. For that reason progesterone or a derivative thereof such as an ester or salt may also be a suitable progestin for use.

The aldosterone-mediated diseases, disorders or conditions of the present invention relates to those diseases that results from the activation of aldosterone receptors (mineralocorticoid receptor) by aldosterone in that this activation has been shown to be a key step in the chain of events leading to a number of diseases originating in the tissue where such receptors are found. Thus, the aldosterone-mediated diseases may be mediated in whole or in part, by aldosterone present in the brain, heart and blood vessels

Examples of aldosterone-mediated diseases that may be treated by the uses and therapeutic methods of the invention are cardiovascular diseases; renal dysfunction; liver diseases; cerebrovascular diseases; vascular diseases; retinopathy; neuropathy; insulinopathy; edema; endothelial dysfunction; baroreceptor dysfunction; migraine headaches. Hypertension may also be an aldosterone-mediated disease, but it is a disease that are thought to be, at least in part, mediated by mineralocorticoid receptors in the kidney and not to the direct activation of aldosterone receptors in the hearth or brain.

In current interesting embodiments of the invention, the aldosterone-mediated disease is a cardiovascular disease. Examples on cardiovascular diseases are heart failure such as congestive heart failure; arrhythmia; diastolic dysfunction, left ventricular diastolic dysfunction, diastolic heart failure, impaired diastolic filling; systolic dysfunction; ischemia; hypertropic cardiomyopathy; sudden cardiac death; myocardial and vascular fibrosis; impaired arterial compliance; myocardial necrotic lesions; vascular damage; myocardial infarction; left ventricular hypertrophy; decreased ejection fraction; cardiac lesions; vascular wall hypertrophy; endothelial thickening; and fibrinoid necrosis of coronary arteries.

As evident to the invention, the women in therapy with the agent exhibiting antimineralocorticoid and progestational effect is healthy, but may nonetheless be susceptible to cardiovascular diseases. The woman to be treated may not has salt sensitivity or an elevated dietary sodium intake or both.

Furthermore, the cardiovascular diseases as mentioned in here may not relate to inflammation-related cardiovascular disorders such as aetherosclerosis and the subject or woman may not need to be in concurrent therapy with ACE-inhibitors, cyclooxygenase-2 inhibitors and/or β-blockers. Of importance is also that the dosage used of the agent exhibiting antimineralocorticoid and progestational effect may not disrupt a patients normal electrolyte and water-retention balance

The preventive effect of an agent exhibiting antimineralocorticoid and progestational on cardiovascular diseases may be investigated in human or animal studies by methods known to the person skilled in the art. In human studies the end-point for determining prevention of cardiovascular diseases may for example be based on the incidences of stroke, incidences of myocardial infarct, incidences of congestive heart failure, incidences of vascular or myocardial fibrosis during long-term studies in a placebo-controlled study or a comparison study involving progestins with substantially no anti-mineralocorticoid effect as the control medication, optionally with concurrent therapy with estrogens. The reduction in the risk of congestive heart failure; myocardial and vascular fibrosis, myocardial infarction and sudden cardiac death is of particular interest to the invention. Indications of cardiovascular diseases may be observed by a physician and through the result of laboratory analyses, such as blood gas analyses or abnormal blood parameters.

A number of animal models for the investigation of the cardiovascular effect of an therapeutically active agent is available. Experiments can be carried out in rodents that are made hypertensive using surgical alterations, or in spontaneously hypertensive rodent treated with a stroke-prone substance.

For example, arterial hypertension can be induced in rodents using different techniques:

a) renovascular hypertension induced by surgically placing a constricting band around the right renal artery, to induce unilateral renal ischemia.

b) infrarenal banding to mechanically constrict blood flow through the aorta below the junction where the renal arteries branch off. (elevated blood pressure in the kidneys.

c) aldosterone infused directly into the rodents at a fixed rate via an implanted pump.

The spontaneously hypertensive rodents of the stroke-prone substrane is characterised by an increased development of severe hypertension, cerebrovascular lesions, and malignant nephrosclerosis.

During the treatment period, the rodents are monitored for hypertension, and plasma aldosterone, systolic and diastolic blood pressure, left ventricular and diastoic pressure, left ventricular dP/dt, body weight, and heart rate.

At the end of the treatment period, the rodents is sacrificed and compared between the different groups of treated with either an active agent or placebo, by examination of the heart, microscopic evaluation of the cerebrovascular damages a histopathologic analysis of the kidneys.

For example, the effect on an agent on cardiocascular injury may be investigated using L-NAME/Ang II/NaCl Hypertensive rats (Delayani et al; eplerenone, a selective aldosterone receptor antagonist. Cardiovascular Drug Reviews vol, 19, No 3, p 185-200).

The mineralocorticoid receptor binding in vivo of an agent may be investigated in adrenolectomized rats by measuring radiolabelled aldosterone bound to receptors in the kidney, hearth, blood vessels, brain or other organs in the presence of the agent. (Delayani et al; eplerenone, a selective aldosterone receptor antagonist. Cardiovascular Drug Reviews vol, 19, No 3, p 185-200).

Other models of Investigating cardiovascular effects are mentioned in: Delyani et al; effect of a selective aldosterone receptor antagonist in myocardial infarction. Am J Physiol Hearth Circ Physiol, vol 50, H647-H654, 2001.

The aldosterone-mediated diseases may also relate to renal dysfunction such as glomerulosclerosis; end-stage renal disease; diabetic nephropathy; reduced renal blood flow; increased gloumerular filtration fraction; proteinuria, decreased gloumerual filtration fraction; decreased creatinine clearance; microalbuminuria; renal arteriopathy; iscemetic lesions; thrombotic lesions; global fribinoid necrosis; focal thrombosis of glomerual capillaries; swelling and prliferation of intracapillary and/or extracapillary cells; expantion of reticulated mesangial matrix with or without significant hypercellularity; and/or malignant nephrosclerosis.

Moreover, liver diseases may be mediated through aldosterone activation of mineralocorticoid receptors. Such liver dieases may be liver cirrhosis, liver ascites or hepatic congestion.

In a particular embodiment, the aldosterone-mediated diseases relate to cerebrovascular disease including stroke.

In further interesting embodiments, the uses and therapeutic actions of the present invention implies treating of vascular diseases such as thrombotic vascular disease; proliferative arteriopathy; atherosclerosis; decreased vascular compliance; and/or endothelial dysfunction.

In current interesting embodiments of the invention, the uses and therapeutic methods of the invention are preferably intended to a peri-menopause woman. That is to say the period before entering menopause. Thus, in interesting embodiments of the invention the woman is in the age from about 40 to 55. The suitable age may also be from about 45 to 53, about 45 to 52, 46 to 52, 48 to 52 or from about 47 to 51 dependent on when the individual women experiences irregular bleeding or other symptoms on deficient endogenous levels of progestogen. However, the uses and therapeutic methods of the invention may not be limited to women of that age. Some women enter the menopause in a much younger age such as for example when they are in theirs thirties due to hypogonadal activities. Other women will enter the menopause upon hysterectomy.

In current preferred embodiments of the invention, the uses and therapeutic methods of the invention is intended to healthy women, e.g. women with a systolic blood pressure less than 140 mm Hg such as less than 130 mm Hg or 120 mm Hg or a diastolic blood pressure less than 105 mm Hg, such as less than 95 mm Hg, 85 mm Hg or 75 mm Hg or combinations of said systolic blood pressure and diastolic blood pressure. An alternative characteristic of a healthy woman relates to the body mass index. Thus, in some embodiments of the invention, the woman has a body mass index in the range from about 16 to 35 in that the body mass index in older woman may be in the higher end of that range without being denoted obesive. Normally, to ensure low risk of cardiovascular diseases, the body-mass index should be lower than 35. Therefore, the body-mass index of a woman is preferably in the range from about 18 to 32, even more preferably in the range from about 18 to 29, most preferably in the range from about 19 to 28, such as most preferably in the range from about 20 to 27.

The uses and therapeutic methods of the invention may be effective in treating aldosterone-mediated diseases in women having risk or even increased risk of cardiovascular diseases such as women with a systolic blood pressure greater than 130 mm Hg or a diastolic blood pressure greater than 85 mm Hg or both. Furthermore, the woman in risk may also be characterised by having activities ratio of plasma aldosterone (ng/dL) to plasma renin (ng/mL/hr) greather than about 30. Women with risk factors can promote the development of arteriosclerosis. Such risk factors are smoking, overweight, stress, genetic disposition, high blood pressure, disorders of lipid metabolism, lack of exercise and/or diabetes.

Furthermore, the uses and therapeutic methods are intended to a woman susceptible to cardiovascular diseases. A subject, such as a woman, may be susceptible to cardiovascular diseases for a number of reasons such as because of hypertension, genetic disorders, race, diabetes, life-style, over-weight, smoking or food-intake. In some embodiments of the invention, the woman is pregnant. In the event where the woman is pregnant the uses for preparation of a medicament and therapeutic methods of the invention comprises an agent exhibiting progestational and anti-mineralocorticoid for the treatment of hypertension in a pregnant woman.

As mentioned, currently interesting embodiments of the invention include that the agent exhibiting progestational and anti-mineralocorticoid effect is drospirenone. In some embodiments of the invention, the dosage of drospirenone is effective in achieving regular and/or bleeding patterns in a woman, e.g. providing cycle control. Such doses may relate to drospirenone in a daily deliverable dose ranging from about 0.02 to 5 mg, preferably ranging from about 0.1 mg to 3 mg or from about 0.25 mg to 3 mg, such as 0.1, 0.25 mg, 0.5 mg, 1, 2 or 3 mg.

In other embodiments of the invention, the dosage of drospirenone would not implicate progestational activity but merely anti-mineralocorticoid effect. In such embodiments, drospirenone is in a daily deliverable dose ranging from about 0.02 to 5 mg, preferably in the range from about 0.1 to 5 mg, more preferably in the range from about 0.1 mg to 3 mg, more preferably in the range from about 0.1 to 2 mg, more preferably in the range from about 0.1 mg to 1 mg, more preferably in the range from 0.1 mg to 0.5 mg, most preferably in the range from about 0.1 mg to 0.4 mg.

Importantly, the dosage of drospirenone or another agent with progestational and antimineralocorticoid effect would not implicate progestational activity such as gynecomastia and/or breast pain in men, or inhibition of ovulation in women. In such event, a still further aspect of the invention relates to the use of drospirenone in a daily deliverable dose of 0.02 to 5 mg for the preparation of a medicament for treating and/or preventing aldosterone-mediated diseases in a subject in need thereof. As may be understood, the subject may preferably be a woman, but not necessarily a woman in peri- or post-menopause. The dose of drospirenone may preferably be in the range from about 0.02 to 5 mg, preferably in the range from about 0.1 to 5 mg, more preferably in the range from about 0.1 mg to 3 mg, more preferably in the range from about 0.1 to 2 mg, more preferably in the range from about 0.1 mg to 1 mg, more preferably in the range from 0.1 mg to 0.5 mg, most preferably in the range from about 0.1 mg to 0.4 mg., such as such as 0.05 mg, 0.1 mg, 0.2 mg, 0.25 mg, 0.3 mg, 0.4 mg, 0.5 mg, or 1 mg. It may further be the understanding that such daily deliverable doses may be for oral administration, orally deliverable doses.

In some embodiments of the invention, drospirenone can be administered in dosages which are effective for preventing myocardial fibrosis but insufficient to substantially increase sodium excretion or substantially reduce potassium retention, reduce hypertension, reduce water retention, affect blood pressure, and/or lower arterial blood pressure. Hence, the daily or monthly deliverable dose of drospirenone may not significantly alter the blood pressure but antagonise the cardiac effects of aldosterone.

As stated, the antialdosteronic effect and progestational effect according to the invention may depend on the dosage of the agent exhibiting antimineralocorticoid effect and progestational effect or on the dosage of the combination of a selective aldosterone antagonist and a progestin with low or substantially no progestational effect. However, the dosing scheme may also have an impact on the therapeutic effect of such agents. The agent according to this invention may be delivered daily to the blood plasma, the mineralocorticoid receptor and/or the progestational receptor for a number of days within each cycle of 21 to 35 days, preferably within each cycle of 28 days. Cycle is intended to mean a period of time that is repeated for every 21 to 35 days. In some embodiments the delivery of the agent may be for at least 5 days up to 35 days. In other embodiments the agent is delivered for at least 10 days, such as 12 days, such as 15 days, such as 20 days, such as 21, 22, 23, 24, 25, 26, 27, or 28 days within each cycle of 21 to 35 days, preferably 28 days. In some embodiments the delivery is in interrupted manner, which means that the agent is delivered for a period of time, which is followed by a period with no agent delivered, which is then followed by a period with delivery of the agent. Also, it means that no agent is delivered for a period of time, followed by delivery of agent, followed by no delivery of agent. This cycling of delivery of an agent may be repeated 1 to 10 times within a cycle of 21 to 35 days. That is to say that each delivery of agent may for example be conducted for 1, 3, 7, 14 or 21 days. The length of the period with delivery of agent may be similar, minor or greater than the period with no delivery. In some embodiments, the agent exhibiting progestational and antimineralocorticoid activity is administered sequentially, which relates to an interrupted manner.

In other embodiments, the agent of the invention, such as an agent exhibiting progestational and antimineralocorticoid activity is administered continuously within a treatment cycle of 21 to 35 days.

The dose delivered by the agent may vary throughout the period of 21 to 35 days or it may be the same. Thus, in some embodiments, the dose delivered varies throughout the period of 21 to 35 days. For example, the dose may be lower in the first half, such as within the first 10 days, of the period of 21 to 35 days than in the second half, such as the last 10 days, of the period of 21 to 35 days. Conversely, it may also be suitable to use a higher dose in the first half, such as within the first 10 days, of the period of 21 to 35 days than in the second half, such as the last 10 days of the period of 21 to 35 days.

The composition according to the invention may be formulated in any suitable manner. In suitable embodiments of the invention, the composition is formulated, according to the person skilled in the art, as a dosage unit for oral or topical administration. Preferable, the composition is formulated for oral delivery of the active agent.

The dosage unit formulated for oral administration may be a solid, semisolid or fluid formulation. The solid dosage units may be selected from the group consisting of uncoated tablets, modified-release tablets, gastro-resistant tablets, orodispersible tablets, effervescent tablets, chewable tablets, soft capsules, hard capsules, modified-release capsules, gastro-resistant capsules, uncoated granules, effervescent granules, coated granules, gastro-resistant granules, modified-release granules, and powders for oral administration; and the fluids are selected from the group consisting of solutions, suspensions or emulsions.

The dosage units for topical administration may be selected from the group consisting of creams, gels, emulsions, suspensions, lotions, suppositories, enemas, pessaries, vaginal capsules, vaginal tablets, pads, sponges, plasters and transdermal delivery systems. The dosage units for parenteral administration may be selected from the group consisting of solutions, suspensions, emulsions, gels, implantation tablets or implants.

Administration of dosage units comprising combinations of active agents such as a selective aldosterone antagonist and a progestin with low or substantially no antimineralocorticoid effect can occur concomitantly or independently. That is to say that the combination is administered in a single dosage form or as more than one dosage form such as one agent administered as a solid and the other agent as a fluid. Similarly, one agent may be formulated for oral administration whilst the other is formulated for transdermal or subcutaneous administration. In a preferred embodiment, both agents are administered as solids formulated for oral administration.

Moreover, an interesting embodiment of the invention comprises a dosage unit wherein the drospirenone is in micronized form or in the form of a cyclodextrin inclusion complex.

The dosage unit of the present invention comprises carriers or excipients, which may act to promote dissolution of both active substances. Examples of such carriers and excipients include substances that are readily soluble in water such as cellulose derivatives, carboxymethyl cellulose, hydroxypropyl cellulose, hydroxypropyl methyl cellulose, gelled starch, gelatin or polyvinylpyrrolidone. In particular, it is anticipated that polyvinylpyrrolidone might be particularly helpful to promote dissolution.

The term “pharmaceutically acceptable carriers and excipients” is intended to mean substances, which are substantially harmless to the individual to which the dosage unit will be administered. Such an excipient normally fulfils the requirements given by the national drug agencies. Official pharmacopeias such as the British Pharmacopeia, the United States of America Pharmacopeia and the European Pharmacopeia set standards for well-known pharmaceutically acceptable excipients.

Suitable pharmaceutically acceptable excipients according to the invention include all kinds that may be used for solid, semi-solid and fluid dosage units.

1. A composition comprising as the sole therapeutically active agent drospirenone or pharmaceutically acceptable salts thereof.

2. The composition according to claim 1, wherein the composition is in unit dosage form and drospirenone is in a dose of 0.02 mg to 5 mg, preferably in a daily deliverable dose of from about 0.1 to 5 mg, more preferably in the range from about 0.1 mg to 3 mg, more preferably in the range from about 0.1 to 2 mg, more preferably in the range from about 0.1 mg to 1.5 mg, even more preferably in the range from about 0.1 mg to 1 mg, most preferably in the range from about 0.1 mg to 0.5 mg, such as from about 0.1 mg to 0.4 mg.

3. The composition according to claim 1, wherein the composition is in unit dosage form and drospirenone is in a dose from about 0.02 mg to 0.4 mg.

4. A method of treating, alleviating or preventing symptoms, diseases or disorders associated with deficient endogenous levels of progesterone comprising administration of an agent exhibiting progestational and antimineralocorticoid activity to a woman.

5. The method according to claim 4, wherein the symptoms, diseases or disorders associated with deficient endogenous levels of progesterone is selected from the group of irregular bleeding; abnormal bleeding; lack of ovulation; risk of miscarriage; peri-menopause and post-menopause comprising the administration of an agent exhibiting progestational and antimineralocorticoid activity.

6. The method according claim 4, wherein said woman is a peri-menopausal woman.

7. The method according to claims 4, wherein said woman is a post-menopausal woman.

8. The method according to claim 4, wherein said woman is in the age from about 40 to 55, preferably from about 45 to 53, more preferably from about 46 to 52, most preferably from about 47 to 51.

9. The method according to claim 4, wherein said woman is pregnant.

10. The method according to claim 4, wherein said woman is in treatment with estrogens.

11. The method according to claim 4, wherein said agent is drospirenone.

12. The method according to claim 11, wherein the drospirenone is in a daily deliverable dose ranging from about 0.02 to 5 mg, preferably ranging from about 0.25 mg to 3 mg.

13. The method according to claim 11, wherein the drospirenone is in a dose intended for a treatment cycle of 21 to 35 days and said dose of drospirenone is from about 3 to 80 mg per cycle, preferably from about 5 mg to 60 mg per cycle, more preferably from about 7 mg to 60 mg per cycle.

14. A method of treating, alleviating or preventing aldosterone-mediated diseases comprising administration of an agent exhibiting progestational and anti-mineralocorticoid activity to a woman that is in need of progesterone replacement therapy.

15. A method of treating, alleviating or preventing aldosterone-mediated diseases, disorders and symptoms associated with deficient endogenous levels of progesterone and simultaneously treating and/or preventing aldosterone-mediated diseases comprising the administration of an agent exhibiting progestational and anti-mineralocorticoid activity to a woman.

16. A method of treating, alleviating or preventing diseases, disorders or symptoms associated with deficient endogenous levels of estrogen and aldosterone-mediated diseases comprising the administration of an estrogen and an agent exhibiting progestational and antimineralocorticoid activity to a woman.

17. A method of treating, alleviating or preventing diseases, disorders or symptoms associated with aldosterone-mediated disease comprising administration of an agent exhibiting progestational and antimineralocorticoid activity to a woman in need of estrogen replacement therapy.

18. A method of treating, alleviating or preventing diseases, disorders or symptoms associated with aldosterone-mediated diseases, comprising administration of drospirenone in a daily deliverable dose of 0.1 to 5 mg to a subject in need thereof.

19. The method according to any one of claims 14 to 18, wherein said agent exhibiting progestational and antimineralocorticoid activity is a non-epoxy spironolactone-type steroidal compound.

20. The method according to claim 18, wherein drospirenone is in a daily deliverable dose ranging from about 0.02 to 5 mg, preferably in the range from about 0.1 to 5 mg, more preferably in the range from about 0.1 mg to 3 mg, more preferably in the range from about 0.1 to 2 mg, more preferably in the range from about 0.1 mg to 1 mg, more preferably in the range from 0.1 mg to 0.5 mg, most preferably in the range from about 0.1 mg to 0.4 mg.

21. The method according to any one of claims 14, 15, 17 and 18, wherein the agent exhibiting progestational and antimineralocorticoid activity is the sole active agent.

22. A method of treating, alleviating or preventing diseases, disorders or symptoms associated with aldosterone-mediated diseases, comprising administration of a combination of a selective aldosterone antagonist and a progestin with low or substantially no anti-mineralocorticoid effect to a woman that is in need of progesterone replacement therapy.

23. A method of treating, alleviating or preventing diseases, disorders or symptoms associated with deficient endogenous levels of estrogen and aldosterone-mediated disease, comprising the administration of an estrogen and a combination of a selective aldosterone antagonist and a progestin with low or substantially no anti-mineralocorticoid effect to a woman.

24. A method of treating, alleviating or preventing diseases, disorders or symptoms associated with aldosterone mediated diseases comprising the administration of a combination of a selective aldosterone antagonist and a progestin with low or substantially no anti-mineralocorticoid effect to a woman in need of estrogen replacement therapy.

25. The method according to any one of claims 22 to 24, wherein the selective aldosterone antagonist is a epoxy spironolactone-type steroidal compound.

26. The method according any one of claims 22 to 24, wherein the selective aldosterone antagonist is selected from the group consisting of eplerenone (epoxymexrenone), mespirenone and canrenoate.

27. The method according any one of claims 22 to 24, wherein the progestin with no antimineralocorticoid effect is selected from the group consisting of progesterone; norethisterone; norethisterone acetat; levonorgestrel; gestodene; norgestilmate; dienogest; medroxyprogesterone acetat; megestrol acetat; chlormadinone acetat; and cyproterone acetat.

28. The use according to claim 27, wherein the progestin with low or substantially no anti-mineralocorticoid effect is progesterone.

29. The method according to claim 15, wherein said deficient endogenous levels of progesterone relate to conditions selected from the group consisting of irregular bleeding; abnormal bleeding; lack of ovulation; risk of miscarriage; peri-menopause and post-menopause.

30. The method according to any one of claims 14 to 18 and 22 to 24, wherein said woman is a peri-menopausal woman.

31. The method according to any one of claims 14 to 18 and 22 to 24, wherein said woman is a post-menopausal woman.

32. The method according to any one of claims 14 to 18 and 22 to 24, wherein said woman is in the age from about 40 to 55, preferably from about 45 to 53, more preferably from about 46 to 52, most preferably from about 47 to 51.

33. The method according to any of one of claims 14 to 18 and 22 to 24, wherein said woman has a body mass index in the range from about 16 to 35, preferably in the range from about 18 to 32, even more preferably in the range from about 18 to 29, most preferably in the range from about 19 to 28, such as most preferably in the range from about 20 to 27.

34. The method according to any one of claims 14 to 18 and 22 to 24, wherein said woman is pregnant.

35. The method according to any of one of claims 22 to 24, wherein said woman is susceptible to said aldosterone-mediated diseases.

36. The method according to claim 34, wherein the woman is hypertensive.

37. The method according to any one of claims 14, 15, 17, 18 and 22 to 24, wherein said woman is in treatment with estrogens.

38. The method according to any one of claims 14 to 18 and 22 to 24, wherein said aldosterone-mediated diseases is selected from the group consisting of hypertension; cardiovascular diseases; renal dysfunction; liver diseases; cerebrovascular diseases; vascular diseases; retinopathy; neuropathy; insulinopathy; edema; endothelial dysfunction; baroreceptor dysfunction; and migraine headaches.

39. The method according to claim 38, wherein said aldosterone-mediated disease is a cardiovascular disease.

40. The method according to claim 38, wherein said cardiovascular disease is selected from the group consisting of heart failure, congestive heart failure; arrhythmia; diastolic dysfunction, left ventricular diastolic dysfunction, diastolic heart failure, impaired diastolic filling; systolic dysfunction; ischemia; hypertropic cardiomyopathy; sudden cardiac death myocardial and vascular fibrosis; impaired arterial compliance; myocardial necrotic lesions; vascular damage; myocardial infraction; left ventricular hypertropy; decreased ejection fraction; cardiac lesions; vascular wall hypertrophy; endothelial thickening; and fibrinoid necrosis of coronary arteries.

41. The method according to claim 38, wherein said cardiovascular disease is myocardial fibrosis.

42. The method according to claim 38, wherein said cardiovascular disease is heart failure.

43. The method according to claim 38, wherein said renal dysfunction is selected from the group consisting of glomerulosclerosis; end-stage renal disease; diabetic nephropathy; reduced renal blood flow; increased gloumerular filtartion fraction; proteinuria, decreased gloumerual filtration fraction; decreased creatinine clearance; microalbuminuria; renal arteriopathy; iscemetic lesions; thrombotic lesions; global fribinoid necrosis; focal thrombosis of glomerual capillaries; swelling and prliferation of intracapillary and/or extracapillary cells; expantion of reticulated mesangial matrix with or without significant hypercellularity; and malignant nephrosclerosis.

44. The method according to claim 38, wherein said cerebrovascular disease includes stroke.

45. The method according to claim 38, wherein said vascular disease is selected from the group consisting of thrombotic vascular disease; proliferative arteriopathy; atherosclerosis; decreased vascular compliance; and endothelial dysfunction.

46. The method according to claim 38, wherein said aldosterone-mediated disease is retinopathy; neuropathy; endothelial dysfunctioin; or baroreceptor dysfunction

47. The method according to claim 14, wherein said woman has symptoms, diseases or disorders associated with deficient endogenous levels of progesterone.

48. The method according to claim 47, wherein said deficient endogenous levels of progesterone relate to conditions selected from the group consisting of irregular ovulation, lack of ovulation, risk of spontaneous abortion, peri-menopause and post-menopause.

49. The method according to claim 38, wherein said effective amount of drospirenone is not capable of reducing the frequency of hot flushes in a post-menopausal woman.

50. The method according to any one of claims 14 to 18 and 22 to 24, wherein the agent exhibiting progestational and anti-mineralocorticoid activity is in a daily deliverable dose that is not capable of reducing the frequency of hot flushes in a post-menopausal woman.

51. The method according to any one of claims 14 to 18 and 22 to 24, wherein the agent exhibiting progestational and anti-mineralocorticoid activity is in a daily deliverable dose that does not significantly altering the blood pressure but antagonise the cardiac effects of aldosterone.

52. The method according to any one of claims 14 to 18 and 22 to 24, wherein the agent exhibiting progestational and anti-mineralocorticoid activity is administered for at least 10 to 35 days within a treatment cycle of 21 to 35 days.

53. The method according to any one of claims 14 to 18 and 22 to 24, wherein the agent exhibiting progestational and anti-mineralocorticoid activity is administered sequentially.

54. The method according to any one of claims 14 to 18, wherein the agent exhibiting progestational and anti-mineralocorticoid activity is administered continuously within a treatment cycle of 21 to 35 days.

55. The method according to any one of claims 14 to 18, wherein the agent exhibiting progestational and anti-mineralocorticoid activity is delivered daily in similar dose.

56. The method according to any one of claims 14 to 18, wherein the agent exhibiting progestational and anti-mineralocorticoid activity is delivered daily in varying doses.

57. The method according to any one of claims 14 to 18, wherein the agent exhibiting progestational and antimineralocorticoid activity is delivered daily in varying doses.
